(19) United States
(12) Patent Application Publication
(10) Pub. No.: US 2012/0122138 A1
(43) Pub. Date: May 17, 2012

(54) CONSUMABLE COMPONENT KIT

(75) Inventors: Nicola Josephine Randles, Staffordshire (GB); Victoria Louise Gray, Newport (GB)

(73) Assignee: Cymtox Limited

(21) Appl. No.: 13/265,199

(22) PCT Filed: Apr. 21, 2010
(86) PCT No.: PCT/GB10/00799

§ 371 Date: Dec. 27, 2011

(30) Foreign Application Priority Data
Apr. 21, 2009 (GB) 0906836.2

(51) Int. Cl. C12M 1/34; C12M 1/12; H01M 8/16; C12Q 1/02; C12N 1/14; C12N 7/00; C12M 1/00; C12N 1/20

(52) U.S. Cl. 435/29; 435/289.1; 435/297.1; 435/288.1; 435/252.1; 435/256.8; 435/235.1; 429/2

(57) ABSTRACT

According to the invention there is provided a consumable component kit for use in a system which utilises a liquid culture of a microbiological material, the kit including: a sealed and aseptic culturing vessel; at least one sealed and aseptic chamber for storing a liquid nutrient medium; a sample of the microbiological material; a sample of the nutrient medium which is suitable for use in a feedstock for the microbiological material; optionally, a sample of salts and other components of the liquid nutrient medium; and a plurality of aseptic conduits for connecting the chamber to the culturing vessel, and for connecting the culturing vessel to the remainder of the system to enable a liquid supply of the microbiological material, or a product or extract thereof, to be utilised by the system.

This invention relates to a consumable component kit for use in a system which utilises a liquid culture of a microbiological material, associated systems, and a method of providing a liquid supply of said microbiological material or a product or an extract thereof.

Fermentors and bioreactors can be used to provide an aqueous supply of microbiological material, such as microorganisms and cells, or products derived from such material. In both cases, sizes can range from micro litre to the cubic meter scale. In general terms, a fermentor is a device which provides an optimal environment for microbiological material to grow in, and is used to yield the desired organism in a suitable state and in a required volume. A bioreactor is a vessel which is used to contain a chemical or biochemical process which involves organisms or biochemically active substances derived from such organisms. Generally speaking, a bioreactor uses the microbiological material to produce a desired product. There are many detailed configurations for both continuous and batch culturing, and these are often configured for specialist applications (see, for example, Pooley et al, Biosensors and Bioelectronics 19 (2004) 1457-1463).

It is a feature and normally a requirement of fermentation processes and bioreactor systems that the culturing process is not compromised by contamination, i.e. by the ingress of unwanted material of a chemical or microbiological nature. It is also necessary to contain the organism or its products in situations where they may be harmful. For this reason strict control of the culture and the maintenance of aseptic conditions is normally a prerequisite for such systems. The present invention satisfies such requirements.

It is known to monitor water quality, in particular to monitor for the presence of contaminants in a water supply, by way of observing the effect of the water supply on a population of bacteria. For example, we have developed a system for continuous monitoring of water contaminants which uses bio-luminescent bacteria such as Vibrio fischeri. Further information concerning this system can be found in our International patent publication WO2006/125954, the entire contents of which are herein incorporated by reference. This system utilises a continuous culture bioreactor or fermentor wherein the bacteria used for testing purposes are harvested in a vessel. To maintain the culture over a period of time requires continual feeding of the bacteria with a mix of salts, buffers, nutrients and air. Additionally, waste products have to be continuously removed, and the system has to be maintained in a condition in which, other than for the presence of the desired bacteria, the system is aseptic. This is because any foreign bacteria which enter the system will compete with the test bacteria for nutrients, and will likely have a deleterious effect on the measurements made by the system. Furthermore, light emitting bacteria tend to reproduce at a low rate compared to other bacteria present in the natural environment, and consequently any contaminant bacteria is likely to lead to the luminescent bacteria being killed off completely within a few days.

Typically, a culture of bio-luminescent bacteria will only be viable for about a month, even if these conditions are maintained assiduously. This is due to bacterial mutation leading to the production of “dark mutant” forms which do not produce light, and eventually predominate in the culture. Thus, after about one month of continuous operation, it is necessary to provide a new, viable bacterial culture in the fermentor. At the present time, this process is performed in laboratory conditions, and requires a high degree of care and best micro-biological practice to avoid contamination of any part of the system, such as the nutrient supply, the fermentor, and associated tubing.

Typically, the bacterial culture is prepared from living culture/agar plates, or from freeze dried (lyophilised) stock. The former method is not a practical one for situations outside of a laboratory. In the latter method, the bacteria have to be stored frozen, or at room temperature in vacuum-packed vials which are expensive to produce. Bacteria must be introduced into the fermentor aseptically, and the fermentor itself must be sterile prior to inoculation with bacteria. Additionally, the nutrient supply and any connections communicating with the fermentor must be sterile. The nutrient broth is typically prepared by dissolving a suitable medium in water with other additives, and then autoclaving the container. The fermentor and associated tubing are normally sterilised by an autoclaving procedure, although other methods may be used. The interconnection of the parts of the system needs to be done with great care in order to maintain sterile conditions, and typically this is done using a flame source and alcohol solution or another chemical agent.

Thus the preparation of a fresh culture requires a high degree of operator skill and is a laborious task. Furthermore, it is possible to envisage many scenarios in which it is undesirable or indeed not possible to prepare a culture in this manner.

The present invention, in at least some of its embodiments, addresses the above named problems. Whilst the invention finds particularly advantageous application in connection with the monitoring of water for contaminants, it can be applied more broadly to the culturing of microbiological material for other purposes.

According to a first aspect of the invention there is provided a consumable component kit for use in a system which utilises a liquid culture of a microbiological material, the kit including:
a sealed and aseptic culturing vessel;
at least one sealed and aseptic chamber for storing a liquid nutrient medium;
a sample of the microbiological material;
a sample of a nutrient medium which is suitable for use in a feedstock for the microbiological material;
optionally, a sample of salts and other components of the liquid nutrient medium; and
a plurality of aseptic conduits for connecting the chamber to the culturing vessel, and for connecting the culturing vessel to the remainder of the system to enable a liquid supply of the microbiological material, or a product or an extract thereof, to be utilised by the system, and, optionally, for connecting the chamber to a supply of the liquid.

In this way a highly convenient culturing system is provided which can be transported to a point of use and easily installed by low-skilled operators. The component kit is aseptic, portable and may be stored in ambient conditions for a period of time before use. As well as permitting convenient preparation of the microbiological material culture, the consumable component kit of the invention can be conveniently coupled to the remainder of the system which utilises the liquid supply of the microbiological material (or a product or an extract thereof) in a facile manner. A further benefit is that maintenance times can be reduced.

Preferably, the chamber is a pouch.

Advantageously, the consumable component kit further includes a disinfecting device for disinfecting a liquid supply into the chamber, the disinfecting device being connected or connectable to an inlet of the chamber. This has the advantage that it is not necessary to provide a supply of sterilised liquid at the point of use.

The microbiological material may be a microorganism or a cell, such as mammalian cells. For the avoidance of doubt, the term “microorganism” includes reference to viruses, prokaryotes (including bacteria and archaea), fungi and protists.

Typically, the liquid is water, and the consumable component kit is usually a system which utilises an aqueous culture of the microbiological material.

The culturing vessel may be a fermentor or a bioreactor. The culturing may be performed under aerobic or anaerobic conditions. Where culturing under aerobic conditions is intended to be performed, the culturing vessel will generally be provided with a suitable inlet allowing control of the intake of air.

The sample of the nutrient medium may be present in concentrated form. This can reduce considerably the weight of the consumable component kit. The sample of the nutrient medium may be provided in dried form, or as a liquid concentrate.

Alternatively, the sample of the nutrient medium may be present in a form directly suitable for use in a feedstock for the microbiological material (eg, the sample may be present as a dilute solution). This is particularly suitable for embodiments in which only small amounts of nutrient media are required, such as mesofluidic or microfluidic embodiments.

Typically, the consumable component kit is a completely disposable item, although some of the components may be recycled after use.

As described in more detail below, use of the consumable component kit includes the preparation of a liquid culture of the microbiological material in the culturing vessel and the preparation of a liquid nutrient medium in the chamber.

Preferably, the sample of the nutrient medium is stored in the chamber. In an alternative embodiment, the sample of the nutrient medium is stored at a location away from the chamber. In this instance, the sample of the nutrient medium would, in use, be dissolved in a liquid supply.

Advantageously, the sample of salts and other components are stored external to the chamber, and contain materials which are hygroscopic and/or of low solubility. Such materials can cause the nutrient medium to clump, which inhibits dissolution of the medium and is aesthetically unpleasing. By separating out materials which are hygroscopic and/or of low solubility and storing the concentrated nutrient medium in the chamber, it is possible to dissolve the nutrient medium in the chamber conveniently at ambient temperature without pre-heating. By “low solubility” it is meant that a material does not dissolve at 20° C. under usual operating conditions within a reasonable period of time without agitation, for example, 30 minutes. The sample of salts and other components which is stored external to the chamber may include hygroscopic constituents and a buffer. For example, the components of a phosphate buffer, which is commonly used in connection with the maintenance of cultures of microorganisms, are hygroscopic, and it has been found to be preferable to store these components external to the chamber and to subsequently introduce them to the chamber as part of an aqueous supply thereto.

Alternatively, the sample of salts and other components of the liquid nutrient medium may be stored with the sample of the nutrient medium in the chamber.

Advantageously, the sample of the microbiological material is in the form of a concentrate, preferably in dried form. Preferably, the sample of the microbiological material is preserved in a preserving matrix. A suitable technique in which microbiological material is preserved in a preserving medium which may be water soluble, is described in UK patent application 2408750, J. Appl. Microbiol. 85 (1998) 913-917 and Letters in Applied Microbiology 41 (2005) 334-340, the entire contents of all of which are herein incorporated by reference. Microorganisms prepared in this way are in a dry form and easily soluble, and therefore can be incorporated readily into the consumable component kit of the present invention. Furthermore, they have the advantages that they can be transported at room temperature and stored at room temperature for up to several days or even weeks without vacuum packaging. Further advantages are that there is typically a high recovery rate of 90% or greater on resuscitation of microorganisms prepared in this way, leading to a more reliable and reproducible microorganism culture. In contrast, freeze dried samples of microorganisms typically have a recovery rate of 10% to 20%. However, the use of other forms of dried samples of microbiological material, such as freeze dried samples, is within the scope of the invention. The use of other systems which encapsulate the microbiological material in a suitable matrix or coating, might be envisaged.

The consumable component kit may further include a sample of sterile resuscitation fluid for use in preparing a liquid culture of the microbiological material in the culturing vessel. Typically, sterile water is utilised, optionally with ionic strength adjusted to suit the strain of microorganism. The sample of sterile resuscitation fluid and the sample of the microbiological material may each be supplied in suitable containers external to the culturing vessel. In use, the sterile resuscitation fluid is introduced to the container containing the sample of the microbiological material under conditions which are, other than for the presence of the desired microbiological material, aseptic, and the resulting solution containing the microbiological material is introduced into the bioreactor.

Alternatively, the consumable component kit may further include a mixing device having a first chamber containing the sample of the microbiological material, a second chamber sealed with the first chamber and containing a sample of sterile resuscitation fluid, and an arrangement operable to place said first and second chambers in fluid communication. The arrangement may comprise a seal which may be broken to place the chambers in fluid communication or a valve which may be opened to place the chambers in fluid communication.

The sample of the microbiological material may be provided separate from the culturing vessel, and may be injected through an injection port.

Alternatively, the sample of the microbiological material may be disposed in a chamber located adjacent to the culturing vessel and separated therefrom by an arrangement operable to place said chamber and the culturing vessel in fluid communication. In these embodiments, the sample of the microbiological material may be disposed adjacent an inlet port of the culturing vessel. In one embodiment, a mixing device is disposed adjacent to the culturing vessel, the mixing device having the first chamber containing the sample of the microbiological material, a second chamber sealed from the first chamber and containing a sample of sterile resuscitation fluid and an arrangement operable to place said first and second chambers in fluid communication. The arrangement may be a seal or a valve as described above.

In further embodiments, the sample of the microbiological material is disposed within the culturing vessel. This offers the further benefit that the microbiological material does not have to be introduced into the culturing vessel at site. The culturing vessel may be used in the preparation of the preserved microbiological material, e.g., the vessel in which the microbiological material is dried is used as the culturing vessel. This can reduce the risk of contamination.

Preferably, the consumable component kit is supplied in an at least partially assembled form. In a partially assembled form, each of the aseptic conduits may be connected to at least one of: the culturing vessel, the chamber, and, if present, the disinfecting device. In a particularly preferred embodiment, the aseptic conduits are fully connected to the culturing vessel, the chamber and, if present, the disinfecting device. In this way, the internal connections within the consumable component kit which are required to provide the liquid supply of the microbiological material are already in place and the kit merely needs to be connected to the remainder of the system.

In alternative embodiments, the consumable component kit includes at least a first assembly and a second assembly, wherein:
the first assembly includes the culturing vessel coupled to one or more conduits, and additionally coupled to a first sealed conduit;
the second assembly includes the chamber coupled to the disinfecting device by one or more conduits, and additionally coupled to a second sealed conduit;
in which the first and second sealed conduits are coupleable so as to connect the chamber to the culturing vessel. Optionally, the first sealed conduit includes a disinfecting device. In these embodiments, the internal interconnections within the consumable component kit may be completed by coupling the first and second sealed conduits. This can be done in a convenient manner by cutting or removing the ends of the first and second sealed conduits and making suitable connections after or during treatment of same with a sterilising medium such as by alcohol spray or immersion. Alternatively, sterile connectors might be employed. The first and second sealed conduits may be sealed with any suitable sterile barrier such as plugs or tape.

Advantageously, the chamber has an aperture which is connected to a multiple way valve arrangement, wherein the multiple way valve arrangement is also in connection with the disinfecting device, and with a conduit which is in connection with or is connectable to the culturing vessel. A three way valve can be used for these purposes. Alternatively the chamber may have an inlet which is in connection or is connectable to the disinfecting device, and an outlet which is in connection or is connectable to the culturing vessel.

Preferably, the pouch is manufactured from a suitable plastics material, such as polyethylene. The skilled reader will appreciate that there are many possible combinations of materials and wall thicknesses which will provide the desired properties, such as low permeability and suitability for use with a desired sterilisation method such as gamma irradiation, autoclaving and treatment with ethylene oxide. Pouches are available commercially as “dry-bags”® from Oxoid Limited, Basingstoke, Hampshire, UK.

The disinfecting device may be a sterile filter, a device for applying localised heat or for irradiating an area or other suitable point disinfecting device. Alternatively, the disinfecting device may be a membrane. The disinfecting device avoids any need for high purity liquid to be provided at site, and aids use at, for example, remote locations.

Typically, the conduits are in the form of tubing, such as flexible tubing. Typically, the conduits are intended for use in a pumping system, which for microbiological purposes is generally a peristaltic pumping system or comprises syringe pumps. However, in principle the invention might be implemented with other forms of conduits and/or used in conjunction with other liquid moving systems. The conduits may incorporate one or more pump heads for facilitating connection to a pumping system such as a peristaltic pumping system.

The consumable component kit may further include a membrane positioned or positionable to control the supply of the liquid to the chamber. The membrane may be positioned external to the chamber and in fluid communication therewith. Alternatively, the membrane may be disposed in or form part of the chamber. The membrane may be formed from a semi-permeable material, and may be a forward osmosis membrane. This might reduce or even obviate a requirement to pump a liquid supply into the chamber in order to generate a liquid nutrient medium, and may reduce handling and equipment costs, and preparation time. The membrane may be used to prevent microorganisms from gaining entry to the chamber, in which instance a separate point disinfecting device may be dispensed with. The membrane may prevent dissolved components of the liquid supply from entering the chamber, and this may be performed selectively, ie, the membrane prevents certain components of the liquid supply from entering the chamber whilst admitting other components. This may facilitate the use of available water sources to prepare the chamber.

A plurality of chambers may be provided to prevent or reduce the frequency of interchange of the liquid nutrient medium or to facilitate the provision of other additives.

In preferred embodiments, the aseptic conduit for connecting the culturing vessel to the remainder of the system is configured to permit bio-luminescence measurements to be made on the liquid supply passing through said conduit.

The plurality of conduits may include one or more syringe pumps. An assembly of syringe pumps may be provided in order to pump fluids through the assembled kit.

Advantageously, at least one component part of the kit includes a mesofluidic or microfluidic element. Advantages can include one or more of small size, low cost, reduced preparation time and reduced reagent consumption. Generally, the mesofluidic or microfluidic element is formed from or is coated with a bio-compatible material. Preferably, at least one of the aseptic conduits includes a mesofluidic or microfluidic element which defines one or more passageways. Advantageously, the aseptic conduit for connecting the culturing vessel to the remainder of the system includes a mesofluidic or microfluidic element which defines one or more passageways. The conduit for connecting the culturing vessel to the remainder of the system may follow a convoluted path through the mesofluidic or microfluidic element. This can reduce the size of the element and improve optical coupling with a light detector in, for example, embodiments where bio-luminescence is detected. The convoluted path may be a serpentine or otherwise curved path. The mesofluidic or microfluidic element may incorporate one or more lens structures for improving optical coupling with a light detector.

At least one of the culturing vessel and the chamber for storing a liquid nutrient medium may include a mesofluidic or microfluidic element. Low volume elements may be used.

The mesofluidic or microfluidic element may include a plate structure. The plate structure may be formed from a plastics material, silicone or glass.

The mesofluidic or microfluidic element may include an oxygen permeable portion. The oxygen permeable portion may be an oxygen permeable membrane.

According to a second aspect of the invention there is provided a system which utilises a liquid supply of a microbiological material, the system including a consumable component kit according to the first aspect of the invention arranged so as to be able to provide a liquid supply of the microbiological material or a product or an extract thereof to the system. The present invention provides particular improvements when the system is a system for monitoring contaminants in water. The system may be a continuous water monitoring system, although the invention extends to non-continuous monitoring systems.

Bio-luminescence may be measured by the system.

It is envisaged that the present invention may usefully extend to other systems which utilise a liquid culture of a microbiological material. For example, the system may be a microbial fuel cell system, a microorganism production facility, a production facility for products produced by the microbiological material, such as enzymes, antibiotics and insulin, a biosensor detection system, an aquarium or a fish farming water quality conditioning system, or a foodstuff manufacturing facility such as for the preparation of yeast feed stock. It may also be used for seed stock preparation, eg, for environmental applications, such as for oil spill digestion, contaminated land remediation or waste water treatment inoculation.

The nature of the consumable component kit is well suited to small or medium scale manufacturing processes. It may be possible to provide a system for replicating a microorganism “in the field”, for example at or close to a remote location where a rare or exotic microorganism has been discovered. The invention may be used in conjunction with foodstuff manufacturing processes, which might be any process that utilises a microbiological process such as yoghurt culture manufacture. The liquid supply of the microbiological material might include any further desirable components, such as nutrients which are utilised by the system. For example, an aqueous supply which includes microorganisms and further nutrients might be used in conjunction with fish farming or aquaria.

According to a third aspect of the invention there is provided a method of providing a liquid supply of a microbiological material or a product or an extract thereof including the steps of:
providing a consumable component kit according to the first aspect of the invention;
transporting said consumable component kit to a system which utilises said liquid culture of a microbiological material;
connecting said consumable component kit to the system;
arranging the consumable component kit so as to be operable to provide said liquid supply of microbiological material, or a product or extract thereof; and
operating the system to utilise said liquid supply of microbiological material, or product or extract thereof.

The third and fourth steps may be performed interchangeably, or simultaneously.

Whilst the invention has been described above, it extends to any inventive combination of the features set out above, or in the following description, drawings or claims.

Embodiments of consumable component kits and systems in accordance with the invention will now be described with reference to the accompanying drawings, in which:—

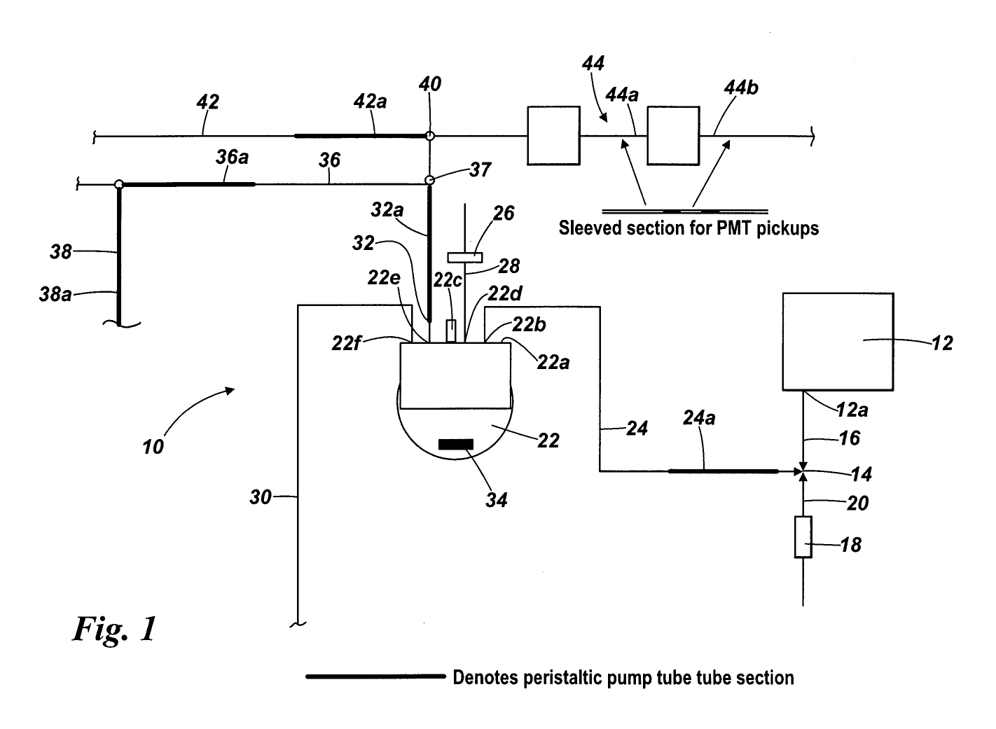

FIG. 1 is a schematic diagram of a first embodiment of an assembled consumable component kit of the invention for use in a system for monitoring water contaminants;

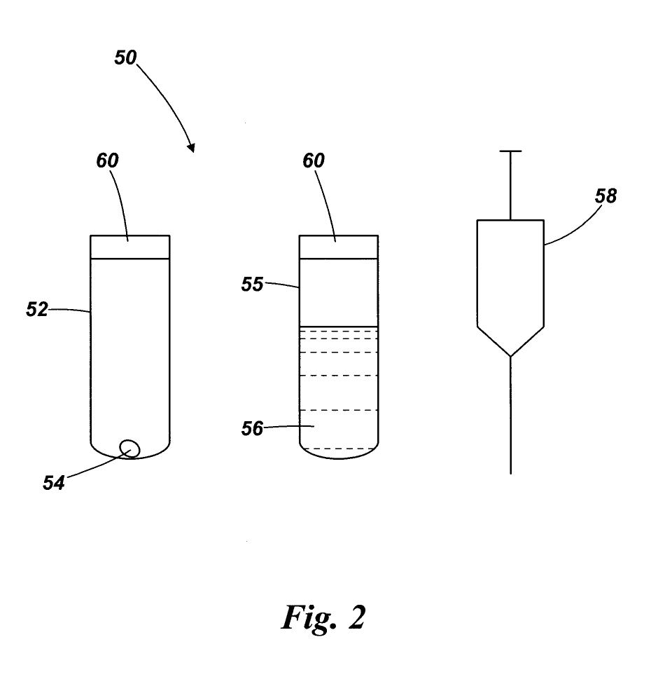

FIG. 2 shows components of a consumable component kit of invention for preparing a bioreagent; and

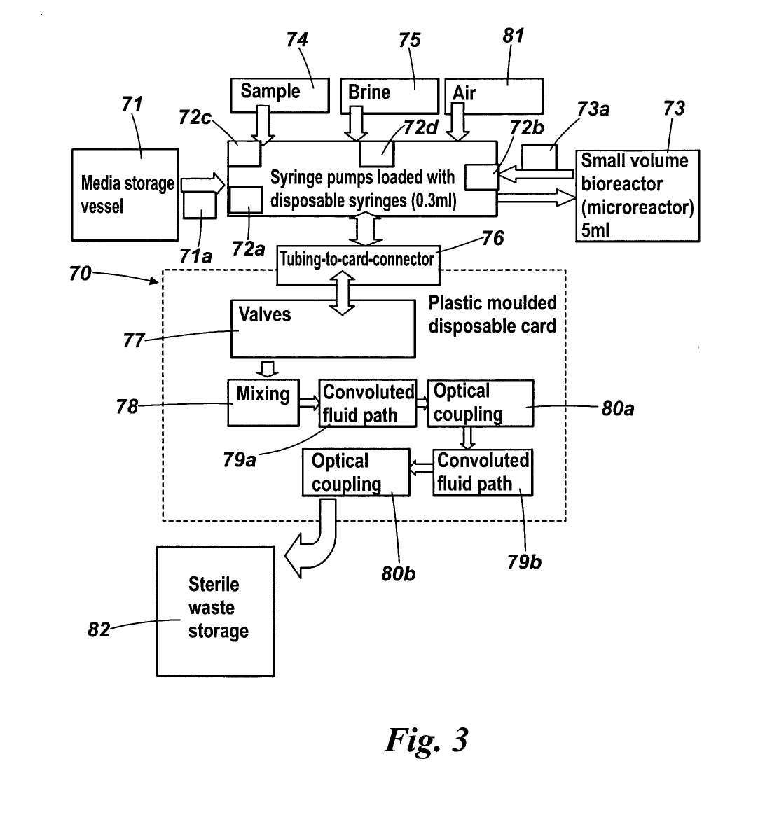

FIG. 3 is a schematic diagram of a second embodiment of an assembled consumable component kit of the invention.

FIG. 1 shows an assembled consumable component kit, depicted generally at 10, for use in a system for monitoring water contaminant. The consumable component kit 10 comprises a flexible pouch 12 having an inlet/outlet 12a. When the consumable component kit is fully assembled and coupled to the monitoring system so as to produce a supply of a bioluminescent bacteria, the flexible pouch contains an aqueous nutrient medium. As described in more detail below, prior to transportation, the nutrient bag is either empty or contains dried products or concentrated liquid products. The nutrient bag 12 is connected to a three way valve 14 by way of tubing 16. A disinfecting filter 18 is connected to the three way valve 14 also by way of tubing 20. Additionally, the three way valve 14 is connected to a fermentor vessel 22 by way of tubing 24 which includes a section of peristaltic pump tubing 24a. The fermentor 22 has a manifold portion 22a which includes an inlet 22b which is in connection with the tubing 24, an inoculation port 22c, an air inlet 22d, a sample line output 22e and a waste outlet 22f. The fermentor can be of any suitable design and construction. Suitable fermentor vessels can be formed from glass, a plastics material such as Perspex, or metal. The manifold portion can be in the form of a lid which is sealed to the main fermentor vessel by way of an O ring. Alternatively, the fermentor and lid assembly can be constructed as a single component. A magnetic stirrer bar 34 is placed inside the fermentor 22.

The air inlet 22d is connected to an air filter 26 by flexible tube 28. The waste outlet 22f is connected to a length of flexible tubing 30 which leads to a suitable waste disposal location, which may advantageously incorporate UV, sterile filtration or other sterilisation means to prevent contamination via the waste line. The bioreagent output 22e is in connection with tubing 32 which includes peristaltic pump tubing 32a. The tubing 32 is connected to a three way connector piece which is also connected to a sample line tube 36 which includes a section of peristaltic pump tubing 36a and may include a section of antimicrobial tubing. The sample line 36 is in turn in connection with a conditioning fluid pump line 38 which includes a section of peristaltic tubing 38a. The output of the three way connector piece 37 is fed (such as via antimicrobiological tubing) to a further three way connector piece 40, which is optionally also in connection with a bubble extraction line 42 which includes a section of peristaltic pump tubing 42a. An air separator/mixing device may be used in place of the three way connector piece. The output of the three way connector piece 40 is in connection with a residence time tubing system 44 comprising an oxygen permeable tube or alternatively a non-oxygen permeable tube used with air segmented flow. This tubing system optionally includes sleeve sections 44a, 44b for photomultiplier tube pickups. Further details concerning the set-up and operation of a continuous water monitoring system can be found in International patent publication WO2006/125954.

Prior to transportation to the site of intended use, the consumable component kit includes a dried sample of the bacteria such as Vibrio fischeri, and a dried sample of the nutrient medium. It is preferable that the dried sample of the bacteria medium is of the type having a preserving matrix. In one embodiment, the dried nutrient medium sample is stored in the flexible pouch 12 and the sample of the bacteria is provided separate to the fermentor 22. FIG. 2 shows a bioreagent preparation kit, depicted generally at 50, which can be used in this embodiment. The bioreagent preparation kit 50 includes a first vial 52 containing a dried sample 54 of the bacteria, and a second vial 55 containing sterile distilled water 56 (or sterile distilled water with components such as salts, buffers and nutrients added). The bioreagent preparation kit 50 further comprises a pre-packed needle and syringe set 58. This avoids having to make a sterile connection from syringe to needle. Both vials 55, 56 have suitable caps 60, such as rubber caps, which can maintain sterility and be penetrated by the needle of the syringe 58. Once at the site of use, the bioreagent can be prepared by inserting the syringe into the second vial 55 and extracting water 56. The first vial 52 is sprayed with alcohol, and water is injected into the first vial 52 by the syringe. The first vial 52 is inverted to mix the contents gently, and the sample of bacteria is left to dissolve with the syringe still connected to the first vial 52. The liquid is then extracted into the syringe 58 and subsequently injected into the fermentor 22 via the inoculation port 22c. The start-up dried nutrient medium is hydrated with distilled or de-ionised water and transferred to the fermentor 22, for example using a large syringe. The liquid start-up medium is then injected into the fermentor 22 via the inoculation port 22c with a filter membrane inserted between the large syringe and the injection needle, allowing the liquid start-up medium to be sterilised prior to entry into the fermentor 22. The liquid in the fermentor is then left to culture under appropriate conditions. In the case of Vibrio fischeri, a culturing period of 24 to 48 hours at a temperature between 10° C. and 28° C. is appropriate. At the lower temperature range of 10 to 15° C. growth is slower and may take longer than three days. A preparation kit can also include the sample of the start-up nutrient medium, the large syringe and filter membrane.

Prior to transportation, the consumable component kit is sterilised to ensure aseptic conditions. Sterilisation is typically achieved by gamma irradiation, although autoclaving or any other suitable method of sterilisation might be employed. It is preferred that the flexible pouch 12, fermentor 22 and all of the associated tubing and other fittings are fully interconnected as shown in FIG. 1 to form a complete assembly which is sterilised prior to transportation. However, it is also possible to provide the consumable component kit as a number of discrete sections which are each sterilised prior to transportation of the entire kit. For example, the flexible pouch 12, disinfecting filter 18, three way valve 14 and associated connections can be formed into a first assembly, the fermentor 22 and associated connections can be formed as a second assembly and the residence time section can be formed as a third assembly, prior to sterilisation. The first, second and third assemblies can be interconnected at a later stage in an aseptic manner. For example, the ends of the assemblies which will be interconnected can be sealed with plugs, autoclave tape or another suitable sterile barrier. These ends can subsequently be removed or cut, and connections made after or during treatment with alcohol by spray or immersion. Alternatively, it may be possible to use sterile connectors to make connections between discrete sub-units of the complete consumable component kit. In another embodiment employing two assemblies, the fermentor and residence time section are connected as a single assembly, with the flexible pouch, disinfecting filter, three way valve and associated connections forming the other assembly.

It is preferred that the flexible pouch 12 is supplied prior to use containing the dried nutrient medium. The pouch may further contain salts and other components of the subsequently produced aqueous nutrient medium. However, it is preferred that components of the aqueous nutrient medium which are of low solubility or are hygroscopic should be supplied external to the flexible pouch 12, Once at the site of intended use, the aqueous nutrient medium is prepared by introducing a stream of water into the flexible pouch 12 via the sterile filter 18. In embodiments in which low solubility and hygroscopic materials are supplied externally to the flexible pouch 12, these materials are pre-dissolved in the supply of water which is introduced to the flexible pouch 12. This has the advantage that the solution of the dried medium contained in the flexible pouch 12 can occur at ambient temperature without any need for pre-heating. An in-line heater can be used to increase the rate of dissolution of the low solubility/hygroscopic materials if required. The components of a phosphate buffer are an example of materials which are hygroscopic which are thus preferred to be supplied externally to the flexible pouch 12. Alternatively, the flexible pouch 12 can be supplied empty, and the entire contents of the nutrient medium is pre-dissolved in water which is then introduced into the flexible pouch 12 through the sterile filter 18. Embodiments in which water or water containing salts and/or other low solubility hygroscopic constituents are pumped is generally preferred to embodiments in which the entire aqueous nutrient medium is pumped into the flexible pouch 12. This is because the former embodiments generally require a lower capacity filter which is economically more attractive.

Once the flexible pouch 12 is full, or a pre-determined amount of water or aqueous solution has been pumped, the relevant pump is switched off. A pressure switch can be included in the flow system in order to trigger an over-pressure condition. Alternatively, a flow meter with a totaliser or load cell can be used to control the amount of liquid dispensed. The flexible pouch 12 and sterile filter 18 are then disconnected from the pump. The pump which is used to fill the flexible pouch 12 may be provided as part of the water monitoring system, in which instance the flexible pouch 12 will already be in place. Alternatively, this pump may be situated at a location close to but separate to the water monitoring system, in which instance the flexible pouch 12 and sterile filter 18 are transferred to the system. The flexible pouch 12 can be conveniently inserted into the system by mounting on a sling or placing in a container. The mounting of the flexible pouch 12 may be performed so that the weight of the flexible pouch 12 is monitored, such as mounting same on a load cell or using a droplet counter in the fermentor vessel. In this way, the feed rate of the aqueous nutrient medium can be controlled 22. This allows the dilution rate of the harvested cells to be controlled in an aseptic manner. In water quality monitoring, maintenance of dilution rate in the water to be tested is important from the point of view of sensitivity.

In this operational mode, the pouch entrance 12a acts as an outlet and the tube 16 takes flow into tubes 24a and 24 through the relevant branch of the three way valve 14. The fermentor 22 is mounted in a suitable housing which contains suitable additional features such as a thermostat, agitator, optical density monitor, photodiode, and other relevant fermentation monitoring devices. The tubing is placed into connection with the remainder of the water monitoring system i.e., peristaltic pump tubing is connected to the peristaltic pumps and the residence time loops may be clipped into place. The tubing can be colour coded in order to assist the connection process. Final connections are made to non-aseptic parts of the water contaminant measurement system using sterile connectors or preferably using straightforward “sterilise, cut and connect” procedures, risk of contamination at these points being minimal.

In alternative embodiments, bioreagent preparation is performed using a different arrangement to that shown in FIG. 2. For example, a snap/mix dispenser device may be used in which the dried bioreagent is contained in one chamber of the device, and a sterile liquid is contained in an adjacent chamber. A seal may be broken or a valve opened to allow the two components to mix. After mixing, the liquid can be injected into the fermentor via the inoculation port. In another embodiment, a snap/mix dispenser device as described above may be mounted directly on the inlet port of the fermentor, with a suitable arrangement being provided to permit the mixed liquid to be introduced into the fermentor. Alternatively, the fermentor may contain sterile nutrient medium, and the microorganism may be injected in reconstituted liquid or dried form directly into the fermentor. Alternatively still, the fermentor may contain the dried bioreagent. In this embodiment, it may be necessary that the atmosphere within the fermentor is under vacuum or dehumidified, and that the fermentor is resistant to the ingress of moisture during storage and/or means are provided within the fermentor to dehumidify the interior of the fermentor during storage. This is because any ingress of moisture during storage can be expected to spoil the bioreagent. The bioreagent might be contained in a soluble pouch, or encapsulated within a soluble coating. Alternatively, the pouch or encapsulating substance may protect against ingress of water, but may release the microorganism on contact with a certain substance, such as an acid or alkali, or under certain conditions.

FIG. 3 is a schematic diagram of a mesofluidic or microfluidic assembled consumable component kit of the invention for use in a system for monitoring water contaminants.

The consumable component kit comprises a mesofluidic or microfluidic assembly 70 and a media storage vessel 71 (which may be rigid or flexible) having an outlet 71a. When the consumable component kit is fully assembled and coupled to the monitoring system so as to produce a supply of, for example, a bioluminescent bacteria, the media storage vessel 71 contains an aqueous nutrient medium. Prior to transportation, the media storage vessel is either supplied with dilute aqueous media or is empty or contains dried or concentrated liquid products and can be prepared as described above. Preferably the media storage vessel is a rigid container which may be a syringe.

Media storage vessel 71 is connected via conduit 71a to a syringe pump 72a loaded with a disposable syringe. The fermentor 73 includes an outlet conduit 73a which is in connection with a syringe pump 72b loaded with a disposable syringe. The fermentor 73 also has an inoculation port, an air inlet, and a waste outlet. The fermentor is a small chamber of glass, plastic or metal, a flexible pouch or preferably is formed in a mesofluidic well plate. This permits the fermentor to be in the form of a microreactor. A representative fermentor volume is about 5 ml, although larger or smaller volumes are possible. The fermentor is preferably pre-loaded with dried microorganisms. Air supply is via an air permeable membrane. The waste outlet flow channel is incorporated into the well plate and connects to a suitable waste disposal location, which may advantageously incorporate UV, sterile filtration or other sterilisation means to prevent contamination via the waste line. A water sample source 74 is in connection with a syringe pump 72c loaded with a disposable syringe. A conditioning fluid source 75 (containing for example, brine) is in connection with a syringe pump 72d loaded with a disposable syringe. The syringe outlet 72b, 72c, 72d are fed via the ‘tubing to card connector’ 76, a valve assembly 77 and inline mixer 78 to a residence time tubing assembly 79a, optical coupling/detection 80a, and then to an optional further residence time tubing assembly 79b and optical coupling/detection 80b. This tubing system optionally includes sleeve sections for photomultiplier tube or silicon photomultiplier pickups, or coupled light guide optical arrangements. Liquid flow is then transferred to a sterile waste store 82. Stirring of the fermentor may be accomplished using a magnetic material in the well which acts as a stirrer on application of an oscillating magnetic field.

The fermentor well and mesofluidic tube plates are manufactured from a rigid substrate such as plastic, silicone or glass incorporating a section or sections of oxygen permeable material or membrane (such as silicone or an ultrathin PTFE film) or allowing for intermittent injection of gaseous air or oxygen or an oxygen carrier. As a thin film structure could potentially be difficult to handle, a sandwich arrangement may be used. In such an arrangement, the fluid path is formed in one plate and a complimentary relief is formed on another plate, with the thin film positioned between the two plates. This permits the construction of a robust structure which allows good oxygenation deep into the device. The plates can be conveniently moulded from a plastic material such as a mouldable fluoropolymer. An example is Teflon PFA®. Other manufacturing techniques might be employed.

The fluidic circuit incorporates a number of passive one-way valves. For each fluid pumped (air 81, conditioning fluid 75, media 71, sample 74, bioreagent 73a) four valves are configured to form a fluidic rectifier. Dead volume associated with the valves is minimised in the design, and different embodiments can include an in-line ball valve or a elastomer valve that inhibits biofilm formation and other biocompatibility issues. The fluid path circuit is designed to be at least partially disposable. Material selection is important in order to prevent biofilm from forming and blocking channels on extended operation.

Where the consumable is part of a monitoring system producing a supply of bioluminescent bacteria, the light is sampled from one or more different observation points along the fluid path. A lens may be moulded into a plastic body to achieve this. The optical properties of the material used for the fluid path should be consistent with the wavelength of the bio-luminescence. The fluid path dimensions may be between 100 and 1000 micron diameter, although conduits of different diameters might be employed. The skilled reader will appreciate that the conduits need not be of circular cross section.

The fluid path circuit can be mass-produced by moulding techniques. This will result in low wall roughness of the fluid path and therefore reduce the potential for biofilm formation. Alternatively, manufacture by machining or etching techniques is possible.

The fluid path is preferably convoluted in order to minimise the overall dimensions of the fluidic card. Convolution may also optimise optical coupling with a light detector and form part of a lens assembly.

The fluid path circuit may contain features found in microreactor technology such as mixing surfaces and optimised connectors. Segmented flow can also be used in the mesofluidic device which improves mixing and also offers additional advantages such as mixing and avoidance of convolution-by-flow effects and maintenance of oxygenation.

Low cost off the shelf interconnects can be used to couple the fermentor well plate and the reagent vessels or flowpaths (media, sample, conditioning fluid) to the syringe pump assembly, from the syringe pump assembly to the fluidic card, and from the fluidic card to the sterile waste storage vessel.

The benefits of the mesofluidic or microfluidic consumable include small size, low cost, reduced preparation time and reduced reagent consumption.

The skilled reader will appreciate that many variations of the embodiments described above are possible. For example, the dried microorganisms may be encapsulated or otherwise coated with various substances which might, for example, permit a phased release of the microorganisms. Microorganisms other than bacteria might be used, as might products of recombinant microbiological procedures. The size of the consumable component kit can be scaled up or scaled down quite significantly, which has the upshot that the present invention can be used in applications requiring relatively small or relatively large scale production of an aqueous supply of microorganisms. As discussed above, microfluidic channels might be used, and additionally, chambers may be used instead of vessels, ie, the entire consumable or parts thereof may be provided as a microfluidic lab on a chip device. The consumable component kit is intended to be used as a true consumable, i.e., once used the consumable component kit is removed from the system and disposed of. However, certain items of the kit or the entire assembly may be recycled for future use. The bioreactor is an example of such a recyclable item. Alternatively, parts of the complete assembly may be formed from biodegradable materials such as biopolymers.

1. A consumable component kit for use in a system which utilises a liquid culture of a microbiological material, the kit including:
a sealed and aseptic culturing vessel;
at least one sealed and aseptic chamber for storing a liquid nutrient medium;
a sample of the microbiological material;
a sample of the nutrient medium which is suitable for use in a feedstock for the microbiological material;
optionally, a sample of salts and other components of the liquid nutrient medium; and
a plurality of aseptic conduits for connecting the chamber to the culturing vessel, and for connecting the culturing vessel to the remainder of the system to enable a liquid supply of the microbiological material, or a product or extract thereof, to be utilised by the system.

2. The consumable component kit according to claim 1 in which the chamber is a pouch.

3. The consumable component kit according to claim 2 in which the pouch is manufactured from a flexible plastics material.

4. The consumable component kit according to claim 1 further including a disinfecting device for disinfecting the liquid supply into the chamber, the disinfecting device being connected or connectable to an inlet of the chamber.

5. The consumable component kit according to claim 1 in which the sample of the nutrient medium is stored in the chamber.

6. The consumable component kit according to claim 5 in which the sample of salts and other components is stored external to the chamber, and contains materials which are hygroscopic and/or of low solubility.

7. The consumable component kit according to claim 1 in which the sample of the nutrient medium is present in concentrated form.

8. The consumable component kit according to claim 1 in which the sample of the microbiological material is in dried form.

9. The consumable component kit according to claim 8 in which the sample of the microbiological material is preserved in a preserving matrix.

10. The consumable component kit according to claim 1 further including a sample of a sterile resuscitation fluid for use in preparing a liquid culture of the microbiological material in the culturing vessel.

11. The consumable component kit according to claim 10 further including a mixing device having a first chamber containing the sample of the microbiological material, a second chamber sealed from the first chamber and containing the sample of sterile resuscitation fluid, and an arrangement operable to place said first and second chambers n fluid communication.

12. The consumable component kit according to claim 1 in which the sample of the microbiological material is provided separate from the culturing vessel.

13. The consumable component kit according to claim 1 in which the sample of the microbiological material is disposed in a chamber located adjacent the culturing vessel and separated therefrom by an arrangement operable to place said chamber and the culturing vessel in fluid communication.

14. The consumable component kit according to claim 1 in which the sample of the microbiological material is disposed within the culturing vessel.

15. The consumable component kit according to claim 4 in which each of the aseptic conduits are connected to at least one of: the culturing vessel, the chamber, and the disinfecting device.

16. The consumable component kit according to claim 11 in which the aseptic conduits are fully connected to the culturing vessel, chamber, and, optionally, the disinfecting device.

17. The consumable component kit according to claim 4 in which the chamber has an aperture which is connected to a multiple way valve arrangement, wherein the multiple way valve arrangement is also in connection with the disinfecting device, and with a conduit which is in connection with or is connectable to the culturing vessel.

18. The consumable component kit according to claim 1 further including a membrane positioned or positionable to control the supply of the liquid into the chamber.

19. The consumable component kit according to claim 18 in which the membrane is a forward osmosis membrane positioned to permit the liquid supply to be supplied to the chamber by forward osmosis.

20. The consumable component kit according to claim 18 in which the membrane acts as a disinfecting device.

21. The consumable component kit according to claim 1 in which the aseptic conduit for connecting the culturing vessel to the remainder of the system is configured to permit bio-luminescence measurements to be made on the liquid supply passing through said conduit.

22. The consumable component kit according to claim 1 in which the plurality of conduits includes one or more syringe pumps.

23. The consumable component kit according to claim 1 in which at least one component part of the kit includes a mesofluidic or microfluidic element.

24. The consumable component kit according to claim 23 in which at least one of the aseptic conduits includes a mesofluidic or microfluidic element which defines one or more passageways.

25. The consumable component kit according to claim 24 in which the aseptic conduit for connecting the culturing vessel to the remainder of the system includes a mesofluidic or microfluidic element which defines one or more passageways.

26. The consumable component kit according to claim 25 in which the conduit for connecting the culturing vessel to the remainder of the system follows a convoluted path through the mesofluidic or microfluidic element

27. The consumable component kit according to claim 23 in which at least one of the culturing vessel and the chamber for storing a liquid nutrient medium includes a mesofluidic or microfluidic element.

28. The consumable component kit according to claim 23 in which the mesofluidic or microfluidic element includes a plate structure.

29. The consumable component kit according to claim 28 in which the plate structure is formed from a plastics material, silicone or glass.

30. The consumable component kit according to claim 23 in which the mesofluidic or microfluidic element includes an oxygen permeable portion.

31. The consumable component kit according to claim 30 in which the oxygen permeable portion is an oxygen permeable membrane.

32. A system which utilises a liquid culture of a microbiological material, the system including a consumable component kit according to claim 1 arranged so as to be operable to provide a liquid supply of the microbiological material or a product or an extract thereof to the system.

33. The system according to claim 32 which is a system for monitoring contaminants in water, preferably a continuous monitoring system.

34. The system according to claim 32 in which bio-luminescence is monitored.

35. The system according to claim 32 selected from: a microbial fuel cell system, a microorganism production facility, a production facility for products produced by the microbiological material, a biosensor detection system, an aquarium or a fish farming water quality conditioning system, and a foodstuff manufacturing facility, and a seed stock preparation facility.

36. A method of providing a liquid supply of a microbiological material or a product or extract thereof including the steps of:
providing a consumable component kit according to claim 1;
transporting said consumable component kit to a system which utilises a liquid culture of a microbiological material;
connecting said consumable component kit to the system;
arranging the consumable component kit so as to be operable to provide a liquid supply of the microbiological material, or a product or an extract thereof; and
operating the system to utilise said liquid supply of the microbiological material, or product or extract thereof.

37. (canceled)
